(19) United States
(12) Patent Application Publication
(10) Pub. No.: US 2026/0351374 A1
(43) Pub. Date: Nov. 5, 2026

(54) ASSESSING PAIN STATES OF PATIENTS AND PROVIDING TREATMENT

(71) Applicant: International Business Machines Corporation, Armonk, NY (US)

(72) Inventors: Jenna Reinen, Rye Brook, NY (US); Carla Paola Agurto Rios, Ossining, NY (US); Guillermo Cecchi, New York, NY (US); Jeffrey L. Rogers, Briarcliff Manor, NY (US); Erhan Bilal, Westport, CT (US)

(21) Appl. No.: 19/194,589

(22) Filed: Apr. 30, 2025

(51) Int. Cl. G16H 20/10; G16H 10/20; G16H 10/60; G16H 50/70

(52) U.S. Cl. CPC G16H 20/10; G16H 10/20; G16H 10/60; G16H 50/70

(57) ABSTRACT

A present invention embodiment assesses pain states of patients. Pain metrics are obtained for a plurality of patients, wherein the pain metrics comprise values for a plurality of dimensions that are each indicative of health outcomes. The pain metrics are analyzed using a clustering model to identify a plurality of clusters. Cluster properties of each of the plurality of clusters are correlated to a corresponding independent pain-related health metric. A treatment is indicated for a particular patient based on the pain metrics of the particular patient corresponding to a particular cluster having a particular pain-related health metric.

### BACKGROUND

### 1. Technical Field

Present invention embodiments relate to patient pain management and machine learning, and more specifically, to assessing pain states of patients by analyzing multidimensional health data using machine learning techniques, and providing treatments based on patients' pain states.

### 2. Discussion of the Related Art

Chronic pain refers to a persistent discomfort or soreness in a patient that lasts for an extended period, typically exceeding three to six months. Chronic pain can be diagnosed based on the magnitude and duration of pain, and can be caused from conditions like arthritis, nerve damage, injuries, or other health conditions. Treatment options for chronic pain can include medications, physical therapy, lifestyle modifications, and in some cases, surgical interventions.

### SUMMARY

According to one embodiment of the present invention, a system is provided for assessing pain states of patients. Pain metrics are obtained for a plurality of patients, wherein the pain metrics comprise values for a plurality of dimensions that are each indicative of health outcomes. The pain metrics are analyzed using a clustering model to identify a plurality of clusters. Cluster properties of each of the plurality of clusters are correlated to a corresponding independent pain-related health metric. A treatment is indicated for a particular patient based on the pain metrics of the particular patient corresponding to a particular cluster having a particular pain-related health metric. Embodiments of the present invention further include a method and computer program product for assessing pain states of patients in substantially the same manner described above.

### BRIEF DESCRIPTION OF THE DRAWINGS

Generally, like reference numerals in the various figures are utilized to designate like components.

FIG. 1 is a diagrammatic illustration of an example computing environment according to an embodiment of the present invention.

FIG. 2 is a block diagram of pain assessment code according to an embodiment of the present invention.

FIG. 3 is an illustration of an environment for assessing patient pain and providing treatment according to an embodiment of the present invention.

FIG. 4 is a radar chart depicting a representation of the multidimensional pain metric data of patients organized by the identified clusters according to an embodiment of the present invention.

FIG. 5 is a table depicting a model indicating correlations between pain metric values and quality of life assessments according to an embodiment of the present invention.

FIG. 6A-6D are diagrams depicting the analysis of pain metric data of a group of patients and classification of patients with respect to a health state according to an embodiment of the present invention.

FIG. 7A-7D are diagrams depicting a process of analyzing patient pain metrics according to an embodiment of the present invention.

FIG. 8 is a flowchart of a method for assessing patient pain and providing treatment according to an embodiment of the present invention.

### DETAILED DESCRIPTION

A present invention embodiment relates to patient pain management and machine learning, and more specifically, to assessing pain states of patients by analyzing multidimensional health data using machine learning techniques, and providing treatments based on patients' pain states.

While chronic pain in patients is conventionally diagnosed by the magnitude and duration of pain, chronic pain is related to other factors such as quality of sleep, mood, medication usage, and/or activity of a patient. Despite these relationships, assessments of chronic pain typically do not consider the breadth of symptoms that impact a patient's health. Recent advances in digital health technology have improved the ability to obtain large sets of data that is potentially relevant for assessing pain, but conventional approaches are not able to apply machine learning techniques to harness this raw data in clinically-meaningful ways. Instead, conventional solutions rely on ten-point pain rating scales without considering other factors that contribute toward a better understanding of a patient's pain experience.

Thus, present invention embodiments provide a novel approach to assessing and treating pain in patients by utilizing unsupervised machine learning techniques that can be assessed to verify that clinically-meaningful results can be obtained. In particular, pain-related health metrics can be collected from patients that describe each patient's mood, activity, medication use, sleep, alertness, analgesia, and other factors, and this data can be provided to an unsupervised clustering model to identify clusters of patients that share similarities with regard to pain in a qualitative and/or quantitative sense. Each cluster can be matched to pain-related health metric for patients, and the values for each of the pain metrics of patients in each cluster can be verified to determine whether particular dimensions of the pain metrics are indicative of a particular pain-related health metric. This enables the dimensionality of the model to be reduced so that only relevant pain metrics are considered for assessing patients, enabling individual patients to be categorized into particular pain states using the data that is relevant. Once patients are diagnosed with respect to a particular pain state, patients can be provided with specific treatments that are optimized for those pain states, thereby improving patient outcomes.

Accordingly, present invention embodiments improve the fields of machine learning and health care by providing a machine learning model that more accurately assesses patients using a holistic metric of health, enabling specific treatments to be selected and provided to patients that are more efficacious. By reducing the dimensionality of the pain metrics that are analyzed, present embodiments reduce the data that is handled at various stages, thereby reducing the amount of computing resources (e.g., processing resources, memory resources, storage resources, and/or networking resources) required to accurately assess patients for pain. Notably, the present embodiments utilize an unsupervised approach that supports fully-automated health care solutions. User feedback can be collected to continuously improve the accuracy of the machine learning model(s) employed over time by updating the models. Thus, present embodiments provide the practical applications of improving the functionality of computing devices in terms of assessing patients for pain while also improving actual health care outcomes of patients.

In some embodiments, correlating the plurality of clusters to the corresponding plurality of pain states includes determining a distance of a centroid for each cluster from a datum in the clustering model, ranking each cluster according to the distance of the centroid to the datum of the independent pain-related health metric, and assigning each cluster to a pain-related health metric according to the ranking. Correlating clusters to pain states in this manner improves the validity of the clustering model for assessing individual patients' pain states, thereby enabling for pain conditions to be treated in a more effective manner.

In some embodiments, the pain state clusters are validated by correlating cluster characteristics, such as centroid distance, to independent pain/health assessments for patients in each cluster. By validating the pain states clusters, overfit or meaningless cluster models can be reduced or eliminated, thereby improving the accuracy of present embodiments in assessing and remediating pain in patients.

In some embodiments, the independent pain assessments are obtained via one or more from a group of: a health care provider, and a natural language processing model that is applied to patient responses to prompts regarding patient well-being. Obtaining pain assessments from a variety of sources improves the richness of data and therefore the accuracy of the clustering model, improving patient outcomes.

In some embodiments, independent pain metrics are determined by analyzing patient feedback that is obtained from the plurality of patients using a questionnaire. By using a questionnaire, patient pain metrics can be collected at the patient's convenience, and can increase the amount of data that is collected from each patient. This also improves the richness of data collected and subsequently, the efficacy of treatments.

In some embodiments, the plurality of dimensions of the pain state cluster metrics includes a mood dimension, an activity dimension, a medication use dimension, a sleep dimension, an alertness dimension, an analgesia dimension, and a wearable sensor dimension. By considering various aspects of a patient's well-being, a more accurate assessment of the patient's pain can be made, thereby improving the efficacy of treatments.

In some embodiments, the treatment is automatically provided to the particular patient using a drug delivery device. In some embodiments, the indicated treatment includes one or more from a group of: a drug administration schedule, and a dosage amount. Thus, present embodiments can provide a fully-automated approach to patient health care that improves patient outcomes.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

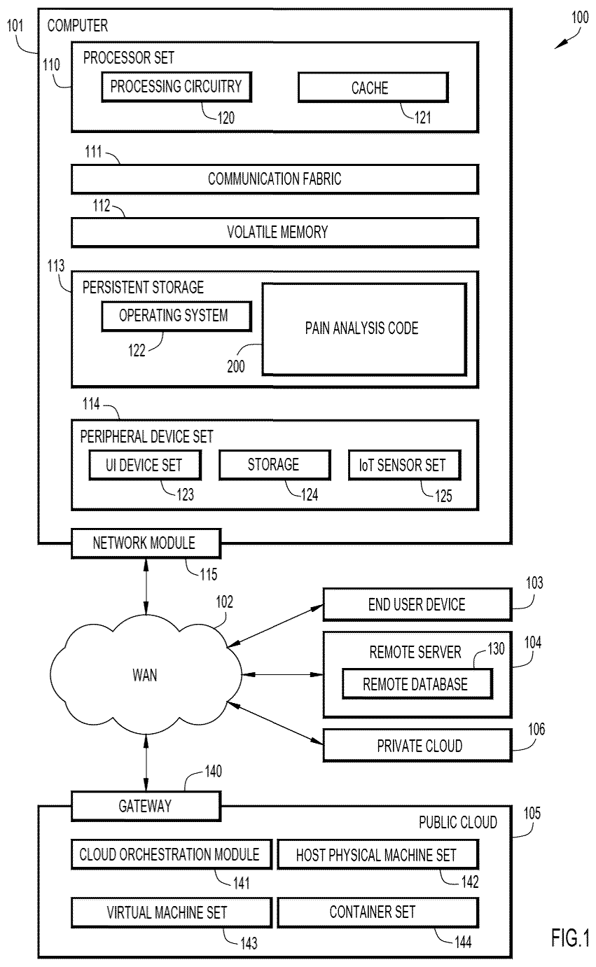

Referring to FIG. 1, computing environment 100 contains an example of an environment for the execution of at least some of the computer code involved in performing the inventive methods, such as pain analysis code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

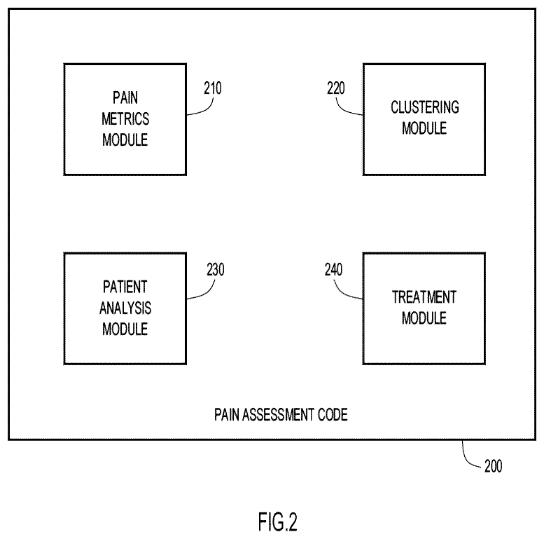

A block diagram of pain analysis code 200 according to an embodiment of the present invention is illustrated in FIG. 2. Specifically, pain analysis code 200 includes a pain metrics module 210, a clustering module 220, a patient analysis module 230, and a treatment module 240. Pain metrics module 210, clustering module 220, patient analysis module 230, and treatment module 240 may include one or more modules or units to perform various functions of present invention embodiments described herein. Pain metrics module 210, clustering module 220, patient analysis module 230, and treatment module 240 may be implemented by any combination of any quantity of software and/or hardware modules or units, and may reside within volatile memory 112 of computer 101 for execution by a processor, such as processor set 110.

Pain metrics module 210 may collect pain metrics from one or more patients that may be descriptive of the pain states of those patients, including states related to chronic pain. The pain metric data can be collected from one or more sources, including user-provided feedback, clinical notes, and/or devices carried or worn by patients (e.g., smartwatches, smartphones, etc.). Thus, the pain metric data can be self-reported or provided by a health care practitioner, and/or can include actigraphy data that is measured by electronic devices.

In some embodiments, the pain metrics include metrics spanning various dimensions, including a mood dimension, an activity dimension, a medication use dimension, a sleep dimension, an alertness dimension, an analgesia dimension, and a wearable sensor dimension. The mood dimension can include data descriptive of the mood of a patient, which can be a value within a range that indicates whether the patient is self-reporting, or has been identified by another individual, as having a good mood versus a bad mood. Mood-related questions can be posed to a patient in a clinical interview, such as a Structured Clinical Interview that is defined by Diagnostic and Statistical Manual of Mental Disorders (DSM) procedures. The activity dimension can include data describing activities performed by a patient, such as specific activities and frequency thereof (e.g., going to work, bathing, cooking, providing care for others, household chores, etc.) and/or movement activities, such as exercise frequency and duration, self-reported movement activity (e.g., time standing per day, number of steps taken per day, etc.). The medication use dimension can include data that describes the frequency and/or amount of dosages of any medications that the patient is taking, including analgesic medication (e.g., non-steroidal anti-inflammatory drugs (NSAIDs), adjuvant medications, opioids, antidepressants, and/or any other drugs prescribed to a patient or relating to a patient's health condition(s). The sleep dimension can include data that describes a patient's duration of sleep, quality of sleep, and the like, or by a bed sensor or accelerometer that estimates sleep staging. The alertness dimension can include data that describes how alert a patient is, including a self-reported description of a patient's cognitive state (e.g., sharp, foggy-headed, etc.), and/or the alertness dimension can be assessed by a clinician based on a patient's responsiveness to questions or conversation, whether or not a patient reacts to stimuli (e.g., opening eyes when their name is called, thigmotaxis, thermotaxis, phototaxis, etc.), and the like. The analgesia dimension can include a patient's subjective sensation of pain, including self-reported pain or pain that is assessed by a clinician. The analgesia dimension can include data descriptive of the intensity of pain, location(s) of a patient's body at which pain is sensed, and the like. The wearable sensor dimension can include any objective biosignal data obtained from a patient, such as resting heart rate, walking heart rate, other types of heart rates (e.g., during exercise), breaths per minute, body temperature, number of steps taken, time spent standing, sleep duration, time spent in various sleep stages, and/or any other relevant data. In some embodiments, the wearable sensor dimension data can be used as data for the other dimensions where relevant.

The pain metrics data for each dimension can be quantified based on predetermined rules that enable scoring of the various dimensions. Any features of a patient's mood data, activity data, medication use data, sleep data, alertness data, analgesia data, and wearable sensor data that indicate that the user is experiencing a higher degree of pain can be scored, whereas any of those features that indicate that the user is experiencing a lower degree of pain, or no pain, can also be scored according to a range of values that evaluate identified clusters of patients with respect to quality of life or disability, which can be an independent functional measure. The predetermined rules can assign values to any qualitative and/or quantitative features of the pain metrics data, and there may be specific rules that are employed for each dimension of the paint metrics data. In some embodiments, weights can be applied to different scores so that some features in the pain metrics data cause the values for each dimension to be influenced to a greater or lesser degree. In some embodiments, self-reported patient responses and/or clinical notes can be provided to one or more natural language processing models that are trained to process unstructured data in order to identify values for each dimension of the pain metrics data. Thus, pain metrics module 210 may process raw pain metrics data collected from patients in order to output values for each assessed dimension of a patient's pain metrics.

Clustering module 220 may include one or more machine learning models that are trained to identify clusters of patients that are similar with respect to the patients' pain metrics. Initially, the values for each dimension of the pain metrics can be normalized with respect to each other (e.g., to a same scale of zero through one, with zero being indicative of a highest pain state and one being indicative of a lowest pain state). The values of assessed dimensions for a plurality of patients can be provided to a clustering model that is trained to identify clusters of patients based on their pain metrics. The clustering model can be a machine learning-based model or other model, and can include a k-means clustering model, a hierarchical clustering model, a mean shift model, a gaussian mixture model, a fuzzy c-means clustering model, a self-organizing maps model, or any neural network-based machine learning model.

Clustering module 220 can analyze patient pain metrics data, which can include average values for each patient for a same given time window (e.g., a one-hour window, a one-day window, etc.). Clustering module 220 may employ an unsupervised approach to cluster identification. Each cluster of patients that is identified by clustering module 220 may thus include patients who are similar with regard to their pain metrics. For example, there may be five clusters, with each cluster generally experiencing a different level of pain. The centroids of each cluster can be computed by clustering module 220 and compared to a baseline datum (e.g., a value that indicates no pain experienced by a patient) to determine the amount of pain experienced by a given cluster of patients. Thus, the clusters of patients can be evaluated with respect to each other, and can serve as definitions for different patient pain states.

The results of clustering module 220 can be verified using independently-determined health scores for patients in each cluster, for example, function or quality of life. The independently-determined health scores can be normalized over a range that matches the ranges of values for dimensions of the pain metrics in the clustering model, or the clustering model's values can be normalized for a pain scale used by the independently-determined pain scores so that a comparison can be made. Thus, using the centroid distance value for each cluster, a cluster can be compared to the independently-determined pain score (e.g., an average for all patients in the cluster) for that cluster to identify the similarity. If there is a threshold degree of closeness (e.g., determined according to r-values), then the results of the unsupervised clustering model may be deemed to be verified, and each verified cluster can be used for other applications of the model, according to the embodiments presented herein. Thus, clusters can be correlated to and labeled as given states. In one embodiment in which five clusters are identified and verified, the clusters can be assigned states such as (i) a best state (high in mood, sleep, activity, analgesia, medicine avoidance, alertness, and mobility (where applicable)), (ii) a second-best state (high in mood, sleep, activity, analgesia, medicine avoidance, alertness, and low in mobility (where applicable)), (iii) a middle state (high in alertness, moderate in other features), (iv) a second-worst state (low in alertness, moderate in other features), and (v) a worst state (low in all states, including mobility (where applicable)).

Patient analysis module 230 may analyze pain metrics for a given patient in order to assess the patient's pain state and/or to provide treatments that can be selected with particularity based on a patient's determined pain state. Once a clustering model is developed (e.g., by clustering module 220) using pain metric data from a group of patients, an individual patient can be analyzed to determine the patient's pain state based on their pain metrics. In particular, patient analysis module 230 may receive pain metric data for a patient and process the data similarly to clustering module 220 in order to provide the patient's pain metric values for each dimension to the clustering model. Then, a clustering algorithm can determine which of the previously-identified clusters best matches the patient. Since each cluster can be labeled with respect to a pain state, the cluster to which the patient's pain metric values best match can be used to determine the patient's pain state. An individual patient's pain metric data can be obtained as time-series data so that at different points in time, the patient's pain state can be determined, as a patient's classification may change from one pain state to another over the course of time.

Treatment module 240 may indicate treatments for patients in order to treat their pain. Different treatments may be defined for different pain states, as patients in different pain states may respond differently to different treatment options. Based on a given patient's pain state, a particular treatment can be determined according to a predetermined rules-based approach, which can be empirically determined based on historical data (i.e., medical outcomes of other patients in those pain states given the known treatments that were provided to those patients).

In particular, treatments can be selected that are targeted to improve a patient's pain state from a state that is more highly associated with poor outcomes to a state that is less associated with poor outcomes. The treatments can be specifically selected in order to improve any dimensions of a patient's pain metrics that correlate highly with regard to outcomes. These treatments may be prioritized such that treatments are preferentially selected and provided to patients based on those treatments targeting dimensions of pain that a patient's pain metrics indicate as being most contributive toward the patient's outcomes.

Thus, treatments can be provided to improve a patient's outcomes (i.e., reduce pain, improve quality of life, improve functionality, etc.) for a mood dimension, an activity dimension, a medication use dimension, a sleep dimension, an alertness dimension, an analgesia dimension, and the like. In order to improve a patient's mood values, a treatment may include therapy, mindfulness exercises, cognitive behavioral therapies, and the like. Treatments for the activity dimension may include an orthopedic device if the patient is experiencing pain associated with a particular limb or movement thereof. Activity-related treatments can also include physical therapy, medications that target particular areas of the body (e.g., analgesics provided to particular nerve pathways, etc.), surgical procedures to reduce pain associated with a patient's activity, and the like. To improve a patient's medication use dimension, a treatment may include a different medication, modifying a dosage amount of a medication, and/or a different dosage schedule. Treatments for the sleep dimension may include medications such as hypnotics or sedatives administered before sleep, or other treatments that improve sleep hygiene, such as recommending different sleeping habits or different bedding to a patient. In order to improve a patient's alertness, treatments may include treatments similar to sleep-related treatments, as well as treatments that involve administering a different type of analgesic to the patient (e.g., switching from an opioid to non-opioid medication). Treatments to improve a patient's analgesia dimension may include selecting different medications and/or dosages to provide to the patient, or other medicinal treatments that may have a higher efficacy than a current treatment.

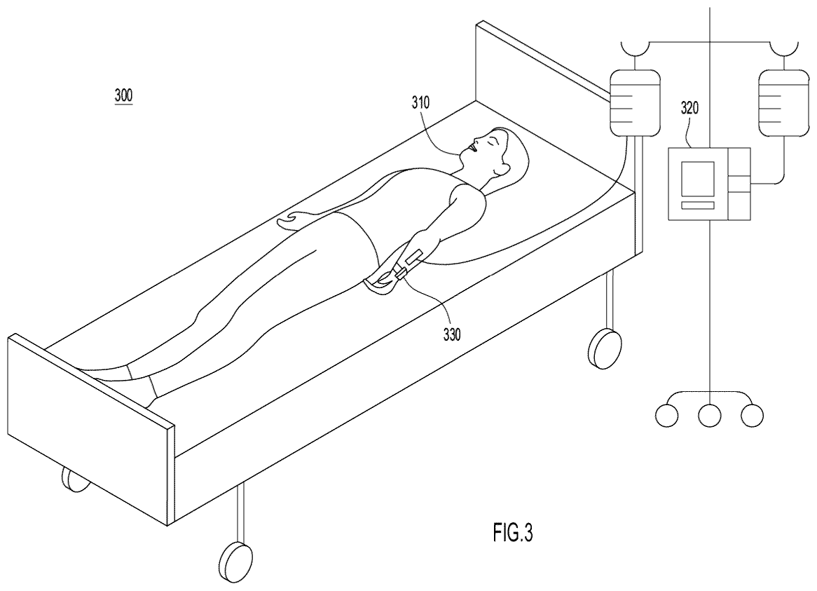

With reference now to FIG. 3, an illustration is provided of an environment 300 for assessing patient pain and providing treatment according to an embodiment of the present invention. As depicted, environment 300 includes a patient 310, a treatment device 320 (e.g., a drug delivery device), and a wearable sensor 330 worn on the patient 310. In addition to any health care metrics that are self-reported by the patient 310 or provided by a health care practitioner, actigraphical data can be collected by wearable sensor 330, which can include a smart watch or other device.

It will be appreciated that the example embodiment of environment 300 is provided for the purpose of describing one manner of automatically providing pain treatment to a patient as described herein. As present embodiments can treat patients having chronic pain, the patients may not be in a bed, but can be ambulatory, and various treatment devices can be worn by a patient or otherwise used by a patient throughout a day, week, month, etc. Thus, present embodiments may provide a fully-automated approach to pain assessment and treatment, enabling the chronic pain of patients to be automatically mitigated.

The patient 310 may be a patient who is being treated for one or more medical conditions, including a condition that causes pain (e.g., chronic pain). Pain metrics may be collected from the patient 310 in order to classify the patient's pain state in accordance with the embodiments presented herein. Thus, based on the pain state of the patient 310, a particular treatment can be provided to the patient in order to treat pain.

Treatment device 320 may include any medical device capable of executing instructions to provide treatment to a patient. Treatment device 320 may including computing and networking components (e.g., a processor, memory, a network interface) so that instructions can be sent to treatment device 320 to cause treatment device 320 to deliver a medical treatment to the patient 310. For example, treatment device 320 may be a pump that can provide an analgesic to patient 310, wherein treatment instructions provided to treatment device 320 indicate a particular dosage amount and/or schedule. Thus, a particular treatment for the patient 310 may be automatically selected based on the particular pain state of the patient 310, and treatment device 320 may automatically provide treatment to the patient 310.

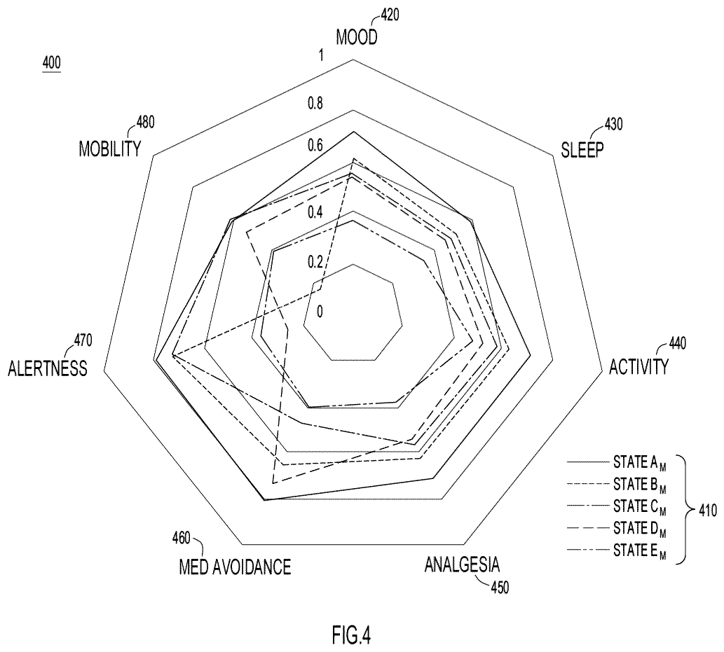

FIG. 4 is a radar chart 400 depicting a specific solution for the multidimensional data from chronic pain patients organized by the identified clusters according to an embodiment of the present invention. As depicted, radar chart 400 includes data for a plurality of pain states 410, including five states Aₘ-Eₘ in the depicted embodiment. The subscript “m” may indicate that the pain metric data was obtained via a wearable device.

Once patient clusters are identified, the average values for each dimension and for each cluster can be represented in radar chart 400. As depicted, the dimensions include a mood dimension 420, a sleep dimension 430, an activity dimension 440, an analgesia dimension 450, a medicine avoidance dimension 460, an alertness dimension 470, and a mobility dimension 480. Data points that are farther from the origin of radar chart 400 may correspond to lower pain states for patients; as such, state Eₘ may represent a worst pain state, whereas state Aₘ may represent a best pain state, with states Bₘ-Dₘ representing states that lay in between the best and worst states. These states are already validated using a measure of quality of life and/or disability, such as the Oswestry Disability Index (ODI), EuroQol 5 Dimensions (EQ-5D) (including the visual analogue scale (VAS)), and the like.

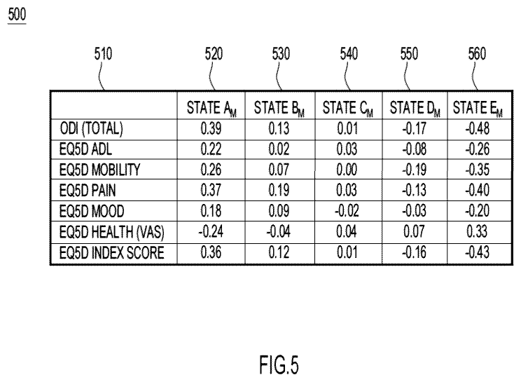

FIG. 5 is a table 500 depicting a model indicating correlations between pain metric values and quality of life assessments according to an embodiment of the present invention. As depicted, table 500 provides r-values that indicate the correlation between cluster centroid distances (e.g., the clusters of FIG. 3) from quality-of-life assessment values that are obtained from a questionnaire. In the depicted embodiment, the quality-of-life assessment values are obtained from a EuroQol 5 Dimensions (EQ-5D) self-report survey that measures quality of life across the dimensions of an Oswestry Disability Index (ODI) (which is holistic representation of patient scores), activities of daily living (ADL), mobility, pain, mood, health (using a visual analogue scale (VAS) with endpoints labeled for best and worst health), and an index score that has values ranging from best to worst health state. Thus, the coefficient values in table 500 indicate correlations between self-reported pain assessments 510 and each cluster of patients 520, 530, 540, 550, and 560. These coefficient values may enable ranking of clusters with regard to whether clusters are “bad” (e.g., a worst pain state) “medium” (e.g., an intermediate pain state), or “good” (e.g., a best pain state).

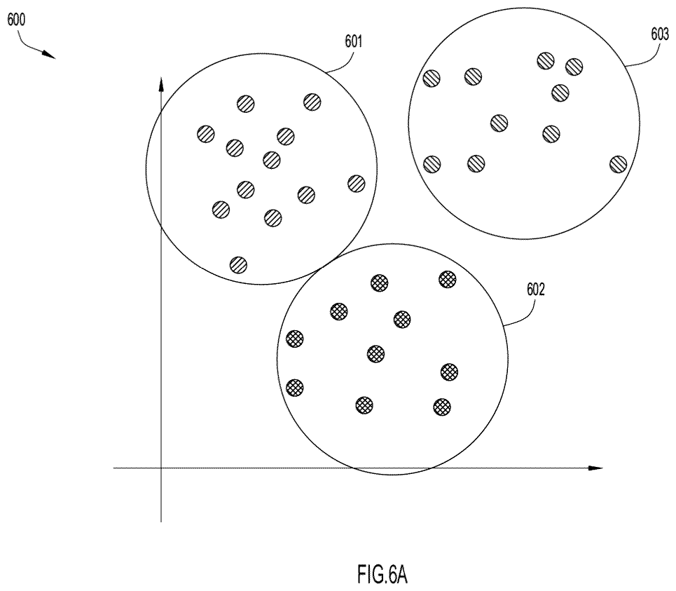

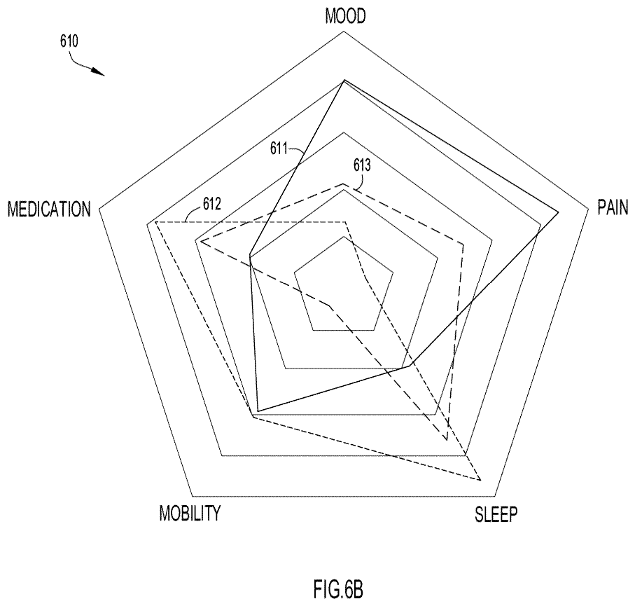

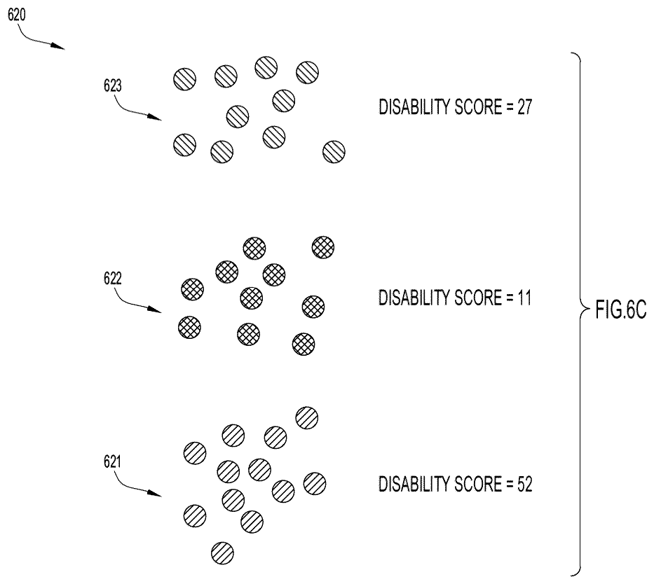

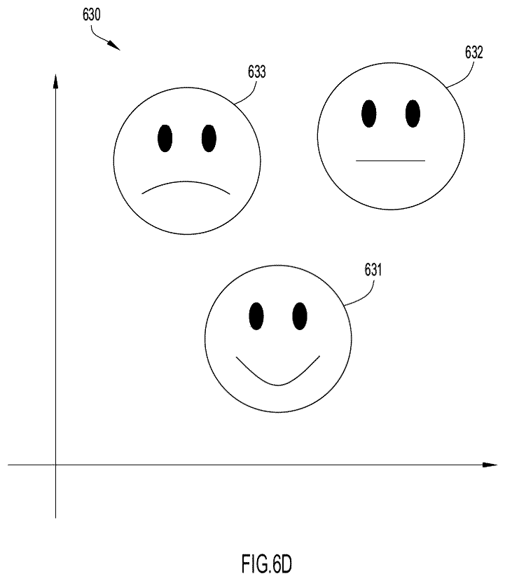

FIG. 6A-6D are diagrams depicting an example of the analysis of pain metric data of a group of patients and classification of patients with respect to a health state according to an embodiment of the present invention.

FIG. 6A is a graph depicting an example of the decomposed representation 600 of patient health care metrics that can be subjected to a k-means clustering analysis. While the depicted embodiment is a two-dimensional graph, it should be appreciated that an n-dimensional space can be used to represent health care metrics across the number of dimensions that are assessed. Thus, the dimensions of decomposed representation 600 may include two different health care metric dimensions for the purpose of describing present invention embodiments. As depicted, three different clusters 601, 602, and 603 are identified.

FIG. 6B is a radar chart 610 depicting an example representation of average pain metric values for the identified clusters of patients in FIG. 6A. Cluster 611 may correspond to cluster 601 of FIG. 1, and similarly, cluster 612 may correspond to cluster 602 of FIG. 1 and cluster 613 may correspond to cluster 603 of FIG. 1. The pain metric data for each cluster can be analyzed by computing the centroid of each cluster, and measuring the distance of each centroid from a datum point (e.g., the center of radar chart 610).

FIG. 6C is a diagram 620 depicting example cluster data for cluster 623 (corresponding to clusters 603 and 613 of FIGS. 6A and 6B), cluster 622 (corresponding to clusters 602 and 612 of FIGS. 6A and 6B), and cluster 621 (corresponding to clusters 601 and 611 of FIGS. 6A and 6B). Each cluster may be correlated to a clinical disability score that is provided for patients in that cluster, which can be an average score of those patients representative of disability and/or quality of life. The correlations can be verified by determining the correlation of measured pain metric values for each cluster with its disability score, and pain states can be assigned, as depicted in FIG. 6D, which is a graph 630 showing a positive pain state 631 for the cluster corresponding to cluster 601, 611, and 621 of FIG. 6A-6C, an intermediate pain state 632 for the cluster corresponding to cluster 602, 612, and 622 of FIG. 6A-6C, and a poor pain state for the cluster corresponding to cluster 603, 613, and 623 of FIG. 6A-6C.

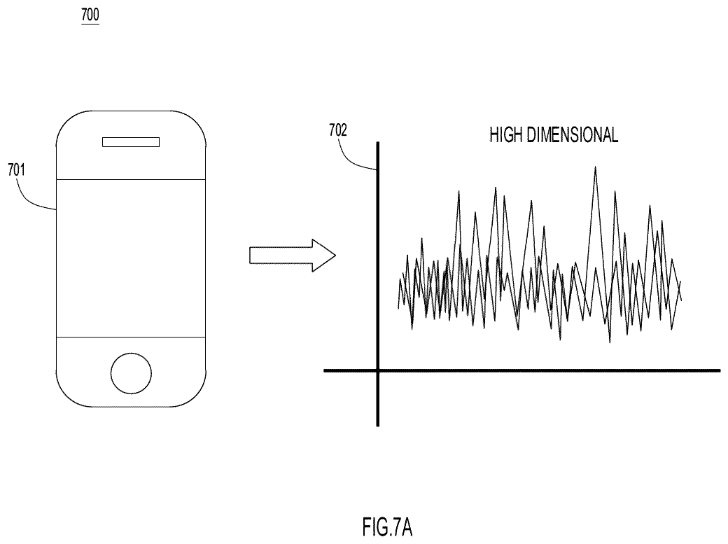

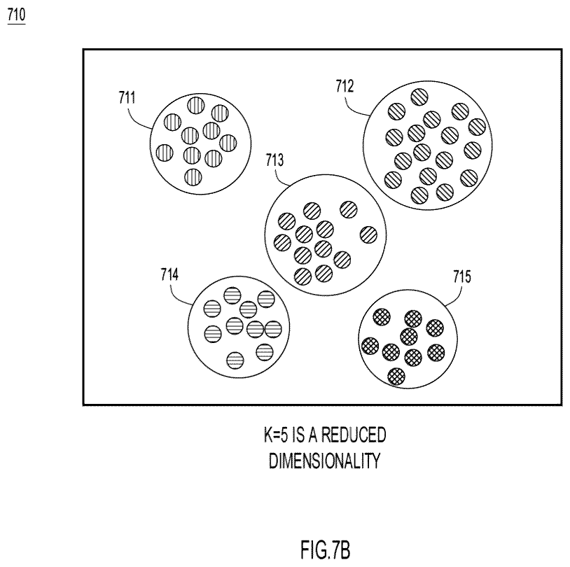

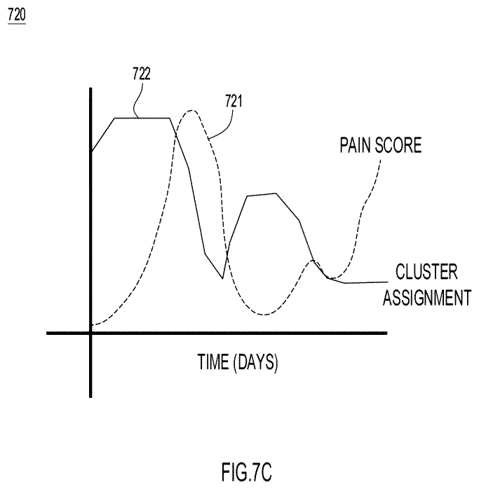

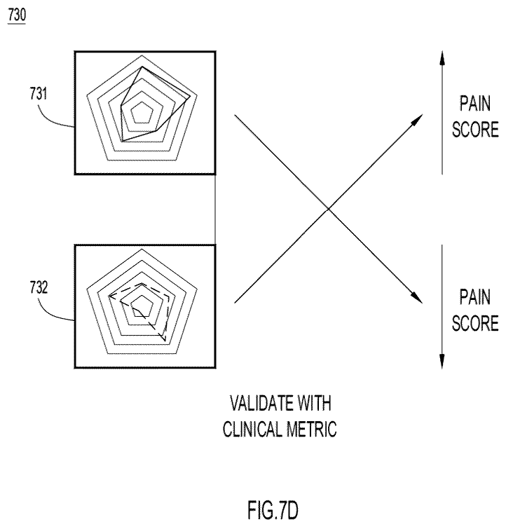

FIG. 7A-7D are diagrams depicting a process of analyzing patient pain metrics according to an embodiment of the present invention.

With reference now to FIG. 7A, a graph 700 is shown depicting an example of the extraction of raw multidimensional pain metric data from patients. The data can be obtained by presenting a questionnaire to a user device 701, such as a smartphone. A patient can input responses to questions in various formats, such as free text entry, selecting multiple choice options, and the like. The resulting data 702 may be unstructured data and/or objective data, such as actigraphy data (e.g., data obtained from a smart watch). The data 702 can be preprocessed to clean the data (e.g., remove outliners), normalize the data across dimensions, and collate the data for a particular time window, as the data 702 can be in a time-series format.

FIG. 7B is a diagram depicting an example clustering model 710 in which the multidimensional pain metrics data of a plurality of patients is analyzed. A clustering algorithm can be applied to identify clusters of patients in the data. In the depicted example, there are five clusters (i.e., k=5), corresponding to cluster 711, cluster 712, cluster 713, cluster 714, and cluster 715. Thus, a reduced dimensionality of the patient pain data can be obtained that indicates how these variable groups relate to each other.

FIG. 7C is a graph 720 depicting example patient pain scores 721 and cluster assignments 722 over time for a selected patient. Clusters can be back-projected onto a patient's timeline so that the trajectory of a patient's pain can be assessed. Thus, a clinician can readily observe when a patient changes from one pain state classification to another over a selected course of time.

FIG. 7D is a diagram 730 showing the validation of clusters 731 and 732 using independently-obtained pain score data. Pairs of cluster assignments and known clinical assessments can be correlated by correlating cluster properties to corresponding independent pain-related health metrics: the centroid distances of each cluster's pain metrics are correlated to known clinical metrics (e.g., pain scores). The values of these comparisons can be evaluated, and clusters can be ranked or otherwise stratified as to whether each cluster represents a “good” or “bad” pain state. In the depicted example, cluster 731 correlates with a lower pain score, and can be classified as a good pain state, whereas cluster 732 correlates with a higher pain score, and can be classified as a bad pain state.

Thus, various clinically-relevant results can be obtained by analyzing patient pain data in accordance with present embodiments. A patient's pain state can be tracked over time, enabling specific treatment options to be provided to the patient in order to mitigate the patient's pain. For example, a patient may be observed to transition from a good pain state to an intermediate pain state before transitioning to a bad pain state. Thus, treatment can be provided to the patient when the patient enters the intermediate pain state in order to prevent the patient from entering the bad pain state.

In some embodiments, patient pain states over time can be provided to a predictive machine learning model, which can be trained to predict future pain states of patients based on their historical pain metric data using a training corpus of examples of such that can be generated in accordance with present embodiments. Thus, a patient can be evaluated to predict future pain state outcomes, which can be used to treat the patient accordingly. For example, if a patient is predicted to enter into a poor pain state in the future, a more aggressive treatment option can be provided to the patient despite the patient's current pain levels not necessarily being indicative of such a treatment.

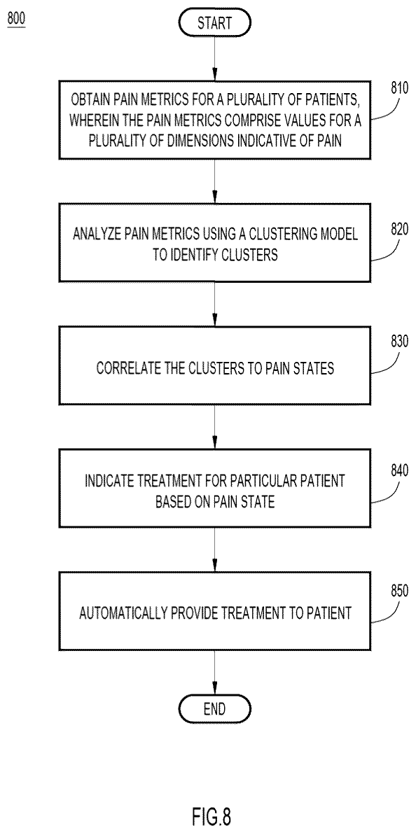

FIG. 8 is a flowchart 800 of a method for assessing patient pain and providing treatment according to an embodiment of the present invention.

Pain metrics for a plurality of patients are obtained at operation 810. The pain metrics can include data that spans a plurality of dimensions that are indicative of pain, and can be obtained from one or more sources. The pain metrics can be self-reported by a patient, or can be obtained from a clinician. In some embodiments, the pain metrics may include actigraphical data that is obtained from a wearable sensor worn by the patient, such as a smart watch. The raw data may be processed with a natural language processing model to obtain values for each dimension that is assessed based on features in the raw data.

The pain metrics are analyzed using a clustering model to identify clusters of patients at operation 820. A clustering model may be applied to identify subsets of patients that are similar to each other based on their pain metric data, and each subset of patients can be assigned into a particular cluster.

The clusters are labeled as particular pain states at operation 830. Using independently-obtained pain scores for the patients, each cluster can be assigned to a particular pain state. Pain metrics for each patient in a cluster can be averaged across a window of time, and these values can be compared to pain scores to provide a pain state for each cluster of patients that designates a particular level of pain. These classifications for each cluster can be validated using clinical health scores of patients to ensure that the classifications are clinically-meaningful.

A treatment is indicated for a particular patient based on the patient's pain state at operation 840. A patient's pain metric data can be provided to the clustering model, and a clustering algorithm can be applied to determine the cluster to which the patient is most similar based on a comparison of the patient's pain metrics to the pain metrics of other patients. Based on the cluster to which the patient is assigned, a particular pain state can be determined for that patient. Next, a specific treatment can be indicated for that patient, which can be selected based on a known efficacy of the treatment for patients in such a pain state.

A treatment is automatically provided to the patient at operation 850. The treatment can be automatically provided by transmitting instructions to a drug delivery device to provide a dosage to a patient at a particular amount and/or according to a particular schedule. Additionally or alternatively, a treatment can be automatically provided by presenting instructions to the patient that instruct the patient to perform (or abstain from) certain habits or activities in order to mitigate pain.

It will be appreciated that the embodiments described above and illustrated in the drawings represent only a few of the many ways of implementing embodiments for automatically assessing patients for pain and providing treatments to patients.

The environment of the present invention embodiments may include any number of computer or other processing systems (e.g., any computing device, treatment delivery device, computing service, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system. These systems may include any types of monitors and input devices (e.g., keyboard, mouse, voice recognition, etc.) to enter and/or view information.

It is to be understood that the software of the present invention embodiments (e.g., pain metrics module 210, clustering module 220, patient analysis module 230, treatment module 240, etc.) may be implemented in any desired computer language and could be developed by one of ordinary skill in the computer arts based on the functional descriptions contained in the specification and flowcharts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client, distributed computing, and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the computing system. The database systems and/or storage structures may be remote from or local to the computer or other processing systems, and may store any desired data.

The present invention embodiments may employ any number of any type of user interface (e.g., Graphical User Interface (GUI), command-line, prompt, etc.) for obtaining or providing information (e.g., pain metric data (including subjective data and/or actigraphy data), patient cluster data, clinical pain score data, pain state data, treatment data, and/or natural language processing data, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

Pain metric data (including subjective data and/or actigraphy data), patient cluster data, clinical pain score data, pain state data, treatment data, and/or natural language processing data may include any information arranged in any fashion, and may be configurable based on rules or other criteria to provide desired information to a user (e.g., a clinician).

The present invention embodiments are not limited to the specific tasks or algorithms described above, but may be utilized for assessing any health state of a patient, including states relating to pain (e.g., chronic pain) but also other states or conditions, including any physical or mental health conditions.

The data may include any format of storing data and may include any data descriptive of pain of a patient. The data may be obtained via any techniques, and may be accessed over a network, fetched from local storage, provided via user input, and the like.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method of assessing pain states of patients comprising:
obtaining pain metrics for a plurality of patients, wherein the pain metrics comprise values for a plurality of dimensions that are each indicative of health outcomes;
analyzing the pain metrics using a clustering model to identify a plurality of clusters;
correlating cluster properties of each of the plurality of clusters to a corresponding independent pain-related health metric; and
indicating a treatment for a particular patient based on the pain metrics of the particular patient corresponding to a particular cluster having a particular pain-related health metric.

2. The computer-implemented method of claim 1, wherein correlating the plurality of clusters to the corresponding plurality of pain states comprises:
determining a distance of a centroid for each cluster from a datum in the clustering model;
ranking each cluster according to the distance of the centroid to a datum of the independent pain-related health metric; and
assigning each cluster to a pain-related health metric according to the ranking.

3. The computer-implemented method of claim 1, wherein the correlating of the plurality of clusters is validated using independent health assessments for patients in each cluster.

4. The computer-implemented method of claim 3, wherein the independent pain assessments are obtained via one or more from a group of: a health care provider, and a natural language processing model that is applied to patient responses to prompts regarding patient well-being.

5. The computer-implemented method of claim 1, wherein the pain metrics are determined by analyzing patient feedback that is obtained from the plurality of patients using a questionnaire.

6. The computer-implemented method of claim 1, wherein the plurality of dimensions of the pain metrics includes a mood dimension, an activity dimension, a medication use dimension, a sleep dimension, an alertness dimension, an analgesia dimension, and a wearable sensor dimension.

7. The computer-implemented method of claim 1, further comprising:
automatically providing the treatment to the particular patient using a drug delivery device.

8. The computer-implemented method of claim 1, wherein the indicated treatment includes one or more from a group of: a drug administration schedule, and a dosage amount.

9. A computer system for assessing pain states of patients comprising:
one or more memories; and
at least one processor coupled to the one or more memories, wherein the at least one processor is configured to:
obtain pain metrics for a plurality of patients, wherein the pain metrics comprise values for a plurality of dimensions that are each indicative of health outcomes;
analyze the pain metrics using a clustering model to identify a plurality of clusters;
correlate cluster properties of each of the plurality of clusters to a corresponding independent pain-related health metric; and
indicate a treatment for a particular patient based on the pain metrics of the particular patient corresponding to a particular cluster having a particular pain-related health metric.

10. The computer system of claim 9, wherein correlating the plurality of clusters to the corresponding plurality of pain states comprises:
determining a distance of a centroid for each cluster from a datum in the clustering model;
ranking each cluster according to the distance of the centroid to the datum of the independent pain-related health metric; and
assigning each cluster to a pain-related health metric according to the ranking.

11. The computer system of claim 9, wherein the correlating of the plurality of clusters is validated using independent health assessments for patients in each cluster.

12. The computer system of claim 11, wherein the independent pain assessments are obtained via one or more from a group of: a health care provider, and a natural language processing model that is applied to patient responses to prompts regarding patient well-being.

13. The computer system of claim 9, wherein the pain metrics are determined by analyzing patient feedback that is obtained from the plurality of patients using a questionnaire.

14. The computer system of claim 9, wherein the plurality of dimensions of the pain metrics includes a mood dimension, an activity dimension, a medication use dimension, a sleep dimension, an alertness dimension, an analgesia dimension, and a wearable sensor dimension.

15. The computer system of claim 9, wherein the at least one processor is further configured to:
automatically provide the treatment to the particular patient using a drug delivery device.

16. The computer system of claim 9, wherein the indicated treatment includes one or more from a group of: a drug administration schedule, and a dosage amount.

17. A computer program product for assessing pain states of patients, the computer program product comprising one or more computer readable storage media having program instructions collectively stored on the one or more computer readable storage media, the program instructions executable by at least one processor to cause the at least one processor to:
obtain pain metrics for a plurality of patients, wherein the pain metrics comprise values for a plurality of dimensions that are each indicative of health outcomes;
analyze the pain metrics using a clustering model to identify a plurality of clusters;
correlate cluster properties of each of the plurality of clusters to a corresponding independent pain-related health metric; and
indicate a treatment for a particular patient based on the pain metrics of the particular patient corresponding to a particular cluster having a particular pain-related health metric.

18. The computer program product of claim 17, wherein correlating the plurality of clusters to the corresponding plurality of pain states comprises:
determining a distance of a centroid for each cluster from a datum in the clustering model;
ranking each cluster according to the distance of the centroid to the datum of the independent pain-related health metric; and
assigning each cluster to a pain-related health metric according to the ranking.

19. The computer program product of claim 17, wherein the correlating of the plurality of clusters is validated using independent health assessments for patients in each cluster.

20. The computer program product of claim 17, wherein the plurality of dimensions of the pain metrics includes a mood dimension, an activity dimension, a medication use dimension, a sleep dimension, an alertness dimension, an analgesia dimension, and a wearable sensor dimension.
